(19) United States
(12) Patent Application Publication
(10) Pub. No.: US 2010/0267067 A1
(43) Pub. Date: Oct. 21, 2010

(54) Synthase Inhibitor Screening Method

(75) Inventor: Jesper Vind, Vaerloese (DK)

Correspondence Address: NOVOZYMES NORTH AMERICA, INC., 500 FIFTH AVENUE, SUITE 1600, NEW YORK, NY, 10110, US

(73) Assignee: Novozymes A/S, Bagsvaerd (DK)

(21) Appl. No.: 12/743,974

(22) PCT Filed: Nov. 26, 2008
(86) PCT No.: PCT/EP08/66219

§ 371 Date: May 20, 2010

(30) Foreign Application Priority Data
Nov. 29, 2007 (EP) 07121855.6

(51) Int. Cl. C12Q 1/48; C12Q 1/02; C12Q 1/26; C12Q 1/34

(52) U.S. Cl. 435/15; 435/29; 435/25; 435/18; 435/23

(57) ABSTRACT

The present invention relates to a method for selecting at least one host cell secreting one or more active enzyme of interest, said method comprising the steps of: a) providing a growth medium comprising one or more synthase inhibitor, which inhibits the synthesis of at least one essential compound in the host cell, and further comprising one or more component, which in the presence of the one or more active enzyme of interest is converted into the at least one essential compound, thereby allowing the host cell to grow; b) cultivating the host cell in or on the growth medium of step (a); and c) selecting at least one host cell capable of growing in or on the growth medium of step (a), which host cell secretes one or more active enzyme of interest.

### SEQUENCE LISTING

The present application comprises a sequence listing.

### FIELD OF THE INVENTION

The present invention relates to selection of host cells, which express an active enzyme of interest under particular growth conditions; the cells which do not express active enzyme under these conditions cannot grow. The synthesis of at least one essential component(s) in the cell is inhibited by one or more synthesis inhibitor added to the growth medium, so the essential component(s) can only be obtained from the medium by the host cell if the active enzyme of interest is being produced.

### BACKGROUND OF THE INVENTION

It has been a goal of commercial enzyme producers to be able to carry out a quick and easy selection method for identifying those host cells that express an active enzyme of interest. Many publications exist that disclose various screening methods, but fewer have provided the means for an actual selection. Most selection-based methods have traditionally employed antibiotic resistance markers. There is a constant need for improved selection methods.

### SUMMARY OF THE INVENTION

In a first aspect, the invention relates to a method for selecting at least one host cell secreting one or more active enzyme of interest, said method comprising the steps of:
a) providing a growth medium comprising one or more synthase inhibitor, which inhibits the synthesis of at least one essential compound in the host cell, and further comprising one or more component, which in the presence of the one or more active enzyme of interest is converted into the at least one essential compound, thereby allowing the host cell to grow;
b) cultivating the host cell in or on the growth medium of step (a); and
c) selecting at least one host cell capable of growing in or on the growth medium of step (a), which host cell secretes one or more active enzyme of interest.

### DETAILED DESCRIPTION OF THE INVENTION

The first aspect of the invention relates to a method for selecting at least one host cell secreting one or more active enzyme of interest, said method comprising the steps of:
a) providing a growth medium comprising one or more synthase inhibitor, which inhibits the synthesis of at least one essential compound in the host cell, and further comprising one or more component, which in the presence of the one or more active enzyme of interest is converted into the at least one essential compound, thereby allowing the host cell to grow;
b) cultivating the host cell in or on the growth medium of step (a); and
c) selecting at least one host cell capable of growing in or on the growth medium of step (a), which host cell secretes one or more active enzyme of interest.

### Host Cells

The present invention also relates to recombinant host cells. A vector comprising a polynucleotide of the present invention is introduced into a host cell so that the vector is maintained as a chromosomal integrant or as a self-replicating extra-chromosomal vector as described earlier. The term “host cell” encompasses any progeny of a parent cell that is not identical to the parent cell due to mutations that occur during replication. The choice of a host cell will to a large extent depend upon the gene encoding the polypeptide and its source.

The host cell may be any cell useful in the recombinant production of a polypeptide of the present invention, e.g., a prokaryote or a eukaryote.

The prokaryotic host cell may be any Gram positive bacterium or a Gram negative bacterium. Gram positive bacteria include, but not limited to, Bacillus, Streptococcus, Streptomyces, Staphylococcus, Enterococcus, Lactobacillus, Lactococcus, Clostridium, Geobacillus, and Oceanobacillus. Gram negative bacteria include, but not limited to, E. coli, Pseudomonas, Salmonella, Campylobacter, Helicobacter, Flavobacterium, Fusobacterium, Ilyobacter, Neisseria, and Ureaplasma.

The bacterial host cell may be any Bacillus cell. Bacillus cells useful in the practice of the present invention include, but are not limited to, Bacillus alkalophilus, Bacillus amyloliquefaciens, Bacillus brevis, Bacillus circulans, Bacillus clausii, Bacillus coagulans, Bacillus firmus, Bacillus lautus, Bacillus lentus, Bacillus licheniformis, Bacillus megaterium, Bacillus pumilus, Bacillus stearothermophilus, Bacillus subtilis, and Bacillus thuringiensis cells.

In a preferred aspect, the bacterial host cell is a Bacillus amyloliquefaciens, Bacillus lentus, Bacillus licheniformis, Bacillus stearothermophilus or Bacillus subtilis cell. In a more preferred aspect, the bacterial host cell is a Bacillus amyloliquefaciens cell. In another more preferred aspect, the bacterial host cell is a Bacillus clausii cell. In another more preferred aspect, the bacterial host cell is a Bacillus licheniformis cell. In another more preferred aspect, the bacterial host cell is a Bacillus subtilis cell.

The bacterial host cell may also be any Streptococcus cell. Streptococcus cells useful in the practice of the present invention include, but are not limited to, Streptococcus equisimilis, Streptococcus pyogenes, Streptococcus uberis, and Streptococcus equi subsp. Zooepidemicus cells.

In a preferred aspect, the bacterial host cell is a Streptococcus equisimilis cell. In another preferred aspect, the bacterial host cell is a Streptococcus pyogenes cell. In another preferred aspect, the bacterial host cell is a Streptococcus uberis cell. In another preferred aspect, the bacterial host cell is a Streptococcus equi subsp. Zooepidemicus cell.

The bacterial host cell may also be any Streptomyces cell. Streptomyces cells useful in the practice of the present invention include, but are not limited to, Streptomyces achromogenes, Streptomyces avermitilis, Streptomyces coelicolor, Streptomyces griseus, and Streptomyces lividans cells.

In a preferred aspect, the bacterial host cell is a Streptomyces achromogenes cell. In another preferred aspect, the bacterial host cell is a Streptomyces avermitilis cell. In another preferred aspect, the bacterial host cell is a Streptomyces coelicolor cell. In another preferred aspect, the bacterial host cell is a Streptomyces griseus cell. In another preferred aspect, the bacterial host cell is a Streptomyces lividans cell.

The host cell may also be a eukaryote, such as a mammalian, insect, plant, or fungal cell.

In a preferred aspect, the host cell is a fungal cell. “Fungi” as used herein includes the phyla Ascomycota, Basidiomycota, Chytridiomycota, and Zygomycota (as defined by Hawksworth et al., In, Ainsworth and Bisby's Dictionary of The Fungi, 8th edition, 1995, CAB International, University Press, Cambridge, UK) as well as the Oomycota (as cited in Hawksworth et al., 1995, supra, page 171) and all mitosporic fungi (Hawksworth et al., 1995, supra).

In a more preferred aspect, the fungal host cell is a yeast cell. “Yeast” as used herein includes ascosporogenous yeast (Endomycetales), basidiosporogenous yeast, and yeast belonging to the Fungi Imperfecti (Blastomycetes). Since the classification of yeast may change in the future, for the purposes of this invention, yeast shall be defined as described in Biology and Activities of Yeast (Skinner, F. A., Passmore, S. M., and Davenport, R. R., eds, Soc. App. Bacteriol. Symposium Series No. 9, 1980).

In an even more preferred aspect, the yeast host cell is a Candida, Hansenula, Kluyveromyces, Pichia, Saccharomyces, Schizosaccharomyces, or Yarrowia cell.

In a most preferred aspect, the yeast host cell is a Saccharomyces carlsbergensis, Saccharomyces cerevisiae, Saccharomyces diastaticus, Saccharomyces douglasii, Saccharomyces kluyveri, Saccharomyces norbensis, or Saccharomyces oviformis cell. In another most preferred aspect, the yeast host cell is a Kluyveromyces lactis cell. In another most preferred aspect, the yeast host cell is a Yarrowia lipolytica cell.

In another more preferred aspect, the fungal host cell is a filamentous fungal cell. “Filamentous fungi” include all filamentous forms of the subdivision Eumycota and Oomycota (as defined by Hawksworth et al., 1995, supra). The filamentous fungi are generally characterized by a mycelial wall composed of chitin, cellulose, glucan, chitosan, mannan, and other complex polysaccharides. Vegetative growth is by hyphal elongation and carbon catabolism is obligately aerobic. In contrast, vegetative growth by yeasts such as Saccharomyces cerevisiae is by budding of a unicellular thallus and carbon catabolism may be fermentative.

In an even more preferred aspect, the filamentous fungal host cell is an Acremonium, Aspergillus, Aureobasidium, Bjerkandera, Ceriporiopsis, Chrysosporium, Coprinus, Coriolus, Cryptococcus, Filibasidium, Fusarium, Humicola, Magnaporthe, Mucor, Myceliophthora, Neocallimastix, Neurospora, Paecilomyces, Penicillium, Phanerochaete, Phlebia, Piromyces, Pleurotus, Schizophyllum, Talaromyces, Thermoascus, Thielavia, Tolypocladium, Trametes, or Trichoderma cell.

In a most preferred aspect, the filamentous fungal host cell is an Aspergillus awamori, Aspergillus fumigatus, Aspergillus foetidus, Aspergillus japonicus, Aspergillus nidulans, Aspergillus niger or Aspergillus oryzae cell. In another most preferred aspect, the filamentous fungal host cell is a Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, or Fusarium venenatum cell. In another most preferred aspect, the filamentous fungal host cell is a Bjerkandera adusta, Ceriporiopsis aneirina, Ceriporiopsis aneirina, Ceriporiopsis caregiea, Ceriporiopsis gilvescens, Ceriporiopsis pannocinta, Ceriporiopsis rivulosa, Ceriporiopsis subrufa, Ceriporiopsis subvermispora, Chrysosporium keratinophilum, Chrysosporium lucknowense, Chrysosporium tropicum, Chrysosporium merdarium, Chrysosporium inops, Chrysosporium pannicola, Chrysosporium queenslandicum, Chrysosporium zonatum, Coprinus cinereus, Coriolus hirsutus, Humicola insolens, Humicola lanuginosa, Mucor miehei, Myceliophthora thermophila, Neurospora crassa, Penicillium purpurogenum, Phanerochaete chrysosporium, Phlebia radiata, Pleurotus eryngii, Thielavia terrestris, Trametes villosa, Trametes versicolor, Trichoderma harzianum, Trichoderma koningii, Trichoderma longibrachiatum, Trichoderma reesei, or Trichoderma viride cell.

### Active Enzyme of Interest

An active enzyme of the present invention may be a polypeptide enzyme obtained from microorganisms of any genus. For purposes of the present invention, the term “obtained from” as used herein in connection with a given source shall mean that the polypeptide encoded by a nucleotide sequence is produced by the source or by a strain in which the nucleotide sequence from the source has been inserted. In another preferred embodiment, the polypeptide obtained from a given source is secreted extracellularly. In a preferred embodiment, the active enzyme of interest is heterologous or homologous.

A polypeptide having enzyme activity of the present invention may be a bacterial polypeptide. For example, the polypeptide may be a gram positive bacterial polypeptide such as a Bacillus, Streptococcus, Streptomyces, Staphylococcus, Enterococcus, Lactobacillus, Lactococcus, Clostridium, Geobacillus, or Oceanobacillus polypeptide having enzyme activity, or a Gram negative bacterial polypeptide such as an E. coli, Pseudomonas, Salmonella, Campylobacter, Helicobacter, Flavobacterium, Fusobacterium, Ilyobacter, Neisseria, or Ureaplasma polypeptide having enzyme activity.

In a preferred aspect, the polypeptide is a Bacillus alkalophilus, Bacillus amyloliquefaciens, Bacillus brevis, Bacillus circulans, Bacillus clausii, Bacillus coagulans, Bacillus firmus, Bacillus lautus, Bacillus lentus, Bacillus licheniformis, Bacillus megaterium, Bacillus pumilus, Bacillus stearothermophilus, Bacillus subtilis, or Bacillus thuringiensis polypeptide having enzyme activity.

In another preferred aspect, the polypeptide is a Streptococcus equisimilis, Streptococcus pyogenes, Streptococcus uberis, or Streptococcus equi subsp. Zooepidemicus polypeptide having enzyme activity.

In another preferred aspect, the polypeptide is a Streptomyces achromogenes, Streptomyces avermitilis, Streptomyces coelicolor, Streptomyces griseus, or Streptomyces lividans polypeptide having enzyme activity.

A polypeptide having enzyme activity of the present invention may also be a fungal polypeptide, and more preferably a yeast polypeptide such as a Candida, Kluyveromyces, Pichia, Saccharomyces, Schizosaccharomyces, or Yarrowia polypeptide having enzyme activity; or more preferably a filamentous fungal polypeptide such as an Acremonium, Agaricus, Alternaria, Aspergillus, Aureobasidium, Botryospaeria, Ceriporiopsis, Chaetomidium, Chrysosporium, Claviceps, Cochliobolus, Coprinopsis, Coptotermes, Corynascus, Cryphonectria, Cryptococcus, Diplodia, Exidia, Filibasidium, Fusarium, Gibberella, Holomastigotoides, Humicola, Irpex, Lentinula, Leptospaeria, Magnaporthe, Melanocarpus, Meripilus, Mucor, Myceliophthora, Neocallimastix, Neurospora, Paecilomyces, Penicillium, Phanerochaete, Piromyces, Poitrasia, Pseudoplectania, Pseudotrichonympha, Rhizomucor, Schizophyllum, Scytalidium, Talaromyces, Thermoascus, Thielavia, Tolypocladium, Trichoderma, Trichophaea, Verticillium, Volvariella, or Xylaria polypeptide having enzyme activity.

In a preferred aspect, the polypeptide is a Saccharomyces carlsbergensis, Saccharomyces cerevisiae, Saccharomyces diastaticus, Saccharomyces douglasii, Saccharomyces kluyveri, Saccharomyces norbensis, or Saccharomyces oviformis polypeptide having enzyme activity.

In another preferred aspect, the polypeptide is an Acremonium cellulolyticus, Aspergillus aculeatus, Aspergillus awamori, Aspergillus fumigatus, Aspergillus foetidus, Aspergillus japonicus, Aspergillus nidulans, Aspergillus niger, Aspergillus oryzae, Chrysosporium keratinophilum, Chrysosporium lucknowense, Chrysosporium tropicum, Chrysosporium merdarium, Chrysosporium inops, Chrysosporium pannicola, Chrysosporium queenslandicum, Chrysosporium zonatum, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Humicola grisea, Humicola insolens, Humicola lanuginosa, Irpex lacteus, Mucor miehei, Myceliophthora thermophila, Neurospora crassa, Penicillium funiculosum, Penicillium purpurogenum, Phanerochaete chrysosporium, Thielavia achromatica, Thielavia albomyces, Thielavia albopilosa, Thielavia australeinsis, Thielavia fimeti, Thielavia microspora, Thielavia ovispora, Thielavia peruviana, Thielavia spededonium, Thielavia setosa, Thielavia subthermophila, Thielavia terrestris, Trichoderma harzianum, Trichoderma koningii, Trichoderma longibrachiatum, Trichoderma reesei, or Trichoderma viride polypeptide having enzyme activity.

It will be understood that for the aforementioned species the invention encompasses both the perfect and imperfect states, and other taxonomic equivalents, e.g., anamorphs, regardless of the species name by which they are known. Those skilled in the art will readily recognize the identity of appropriate equivalents.

Strains of these species are readily accessible to the public in a number of culture collections, such as the American Type Culture Collection (ATCC), Deutsche Sammlung von Mikroorganismen and Zellkulturen GmbH (DSM), Centraalbureau Voor Schimmelcultures (CBS), and Agricultural Research Service Patent Culture Collection, Northern Regional Research Center (NRRL).

Furthermore, such polypeptides may be identified and obtained from other sources including microorganisms isolated from nature (e.g., soil, composts, water, etc.) using the above-mentioned probes. Techniques for isolating microorganisms from natural habitats are well known in the art. The polynucleotide may then be obtained by similarly screening a genomic or cDNA library of such a microorganism. Once a polynucleotide sequence encoding a polypeptide has been detected with the probe(s), the polynucleotide can be isolated or cloned by utilizing techniques that are well known to those of ordinary skill in the art (see, e.g., Sambrook et al., 1989, supra).

Polypeptides of the present invention also include fused polypeptides or cleavable fusion polypeptides in which another polypeptide is fused at the N-terminus or the C-terminus of the polypeptide or fragment thereof. A fused polypeptide is produced by fusing a nucleotide sequence (or a portion thereof) encoding another polypeptide to a nucleotide sequence (or a portion thereof) of the present invention. Techniques for producing fusion polypeptides are known in the art, and include ligating the coding sequences encoding the polypeptides so that they are in frame and that expression of the fused polypeptide is under control of the same promoter(s) and terminator.

A fusion polypeptide can further comprise a cleavage site. Upon secretion of the fusion protein, the site is cleaved releasing the polypeptide having enzyme activity from the fusion protein. Examples of cleavage sites include, but are not limited to, a Kex2 site that encodes the dipeptide Lys-Arg (Martin et al., 2003, J. Ind. Microbiol. Biotechnol. 3: 568-76; Svetina et al., 2000, J. Biotechnol. 76: 245-251; Rasmussen-Wilson et al., 1997, Appl. Environ. Microbiol. 63: 3488-3493; Ward et al., 1995, Biotechnology 13: 498-503; and Contreras et al., 1991, Biotechnology 9: 378-381), an Ile-(Glu or Asp)-Gly-Arg site, which is cleaved by a Factor Xa protease after the arginine residue (Eaton et al., 1986, Biochem. 25: 505-512); a Asp-Asp-Asp-Asp-Lys site, which is cleaved by an enterokinase after the lysine (Collins-Racie et al., 1995, Biotechnology 13: 982-987); a His-Tyr-Glu site or His-Tyr-Asp site, which is cleaved by Genenase I (Carter et al., 1989, Proteins: Structure, Function, and Genetics 6: 240-248); a Leu-Val-Pro-Arg-Gly-Ser site, which is cleaved by thrombin after the Arg (Stevens, 2003, Drug Discovery World 4: 35-48); a Glu-Asn-Leu-Tyr-Phe-Gln-Gly site, which is cleaved by TEV protease after the Gln (Stevens, 2003, supra); and a Leu-Glu-Val-Leu-Phe-Gln-Gly-Pro site, which is cleaved by a genetically engineered form of human rhinovirus 3C protease after the Gln (Stevens, 2003, supra).

Also, in preferred embodiments of the invention, the active enzyme of interest is a lyase, a ligase, a hydrolase, an oxidoreductase, a transferase, or an isomerase, and more preferably the enzyme is an amylolytic enzyme, a lipolytic enzyme, a proteolytic enzyme, a cellulytic enzyme, an oxidoreductase or a plant cell-wall degrading enzyme, and more preferably an enzyme with an activity selected from the group consisting of aminopeptidase, amylase, amyloglucosidase, carbohydrase, carboxypeptidase, catalase, cellulase, chitinase, cutinase, cyclodextrin glycosyltransferase, deoxyribonuclease, esterase, galactosidase, beta-galactosidase, glucoamylase, glucose oxidase, glucosidase, haloperoxidase, hemicellulase, invertase, isomerase, laccase, ligase, lipase, lyase, mannosidase, oxidase, pectinase, peroxidase, phytase, phenoloxidase, polyphenoloxidase, protease, ribonuclease, transferase, transglutaminase, or xylanase.

### Selection of Lipases

Selection of transformants secreting active lipolytic enzyme could be done using any fatty acid synthase inhibitor, such as, triclosan, cerulenin, thiolactomycin or diazaborin. The lipolytic enzyme can be any carboxyl-esterase having activity on ester bonds in substrates such as triglyceride lipid, phospholipid, galactolipid.

### Selection of Proteases

Selection of transformants secreting active proteolytic could by done using any amino acid synthase inhibitor, such as 5-nitro-2-benzimidazolinone, glyphosate, carboxymethoxylamin, O-allylhydroxylamine, indole acrylic acid, O-(carboxymethyl) hydroxylamine hemihydrochloride, imidazolinones, or sulfonyl urea. The amino acid synthesis can also be inhibited by the addition of amino acids. For instance E. coli can be starved for tryptophan by the addition of tyrosine and phenylalanine due to feed back inhibition of the amino acid synthesis route, which is common for the three amino acids. The proteolytic enzyme can be any protease having activity on any type of peptide bond.

### Selection of Carbohydrases

Selection of transformants secreting active carbohydrase could be done using any inhibitor, such as phloretin, pyridoxal phosphate, theilavin A, 2-hydroxy-5-nitrobenzaldehyde, or Mumbaistatin against glucose-6-phophatase; or 2,3-dihydro-1H-cyclopenta[b]quinoline against fructose 1,6 biphosphatase; or amitrole or aptamine against glucosamine-6P synthase. The carbohydrase can be any enzyme cleaving bonds in carbohydrates releasing, for example, glucosamine-6P, glucose or fructose 6-phosphate.

### DNA Introduction

The introduction of DNA into a Bacillus cell may, for instance, be effected by protoplast transformation (see, e.g., Chang and Cohen, 1979, Molecular General Genetics 168: 111-115), by using competent cells (see, e.g., Young and Spizizen, 1961, Journal of Bacteriology 81: 823-829, or Dubnau and Davidoff-Abelson, 1971, Journal of Molecular Biology 56: 209-221), by electroporation (see, e.g., Shigekawa and Dower, 1988, Biotechniques 6: 742-751), or by conjugation (see, e.g., Koehler and Thorne, 1987, Journal of Bacteriology 169: 5271-5278). The introduction of DNA into an E coli cell may, for instance, be effected by protoplast transformation (see, e.g., Hanahan, 1983, J. Mol. Biol. 166: 557-580) or electroporation (see, e.g., Dower et al., 1988, Nucleic Acids Res. 16: 6127-6145). The introduction of DNA into a Streptomyces cell may, for instance, be effected by protoplast transformation and electroporation (see, e.g., Gong et al., 2004, Folia Microbiol. (Praha) 49: 399-405), by conjugation (see, e.g., Mazodier et al., 1989, J. Bacteriol. 171: 3583-3585), or by transduction (see, e.g., Burke et al., 2001, Proc. Natl. Acad. Sci. USA 98: 6289-6294). The introduction of DNA into a Pseudomonas cell may, for instance, be effected by electroporation (see, e.g., Choi et al., 2006, J. Microbiol. Methods 64: 391-397) or by conjugation (see, e.g., Pinedo and Smets, 2005, Appl. Environ. Microbiol. 71: 51-57). The introduction of DNA into a Streptococcus cell may, for instance, be effected by natural competence (see, e.g., Perry and Kuramitsu, 1981, Infect. Immun. 32: 1295-1297), by protoplast transformation (see, e.g., Catt and Jollick, 1991, Microbios. 68: 189-2070, by electroporation (see, e.g., Buckley et al., 1999, Appl. Environ. Microbiol. 65: 3800-3804) or by conjugation (see, e.g., Clewell, 1981, Microbiol. Rev. 45: 409-436). However, any method known in the art for introducing DNA into a host cell can be used.

Fungal cells may be transformed by a process involving protoplast formation, transformation of the protoplasts, and regeneration of the cell wall in a manner known per se. Suitable procedures for transformation of Aspergillus and Trichoderma host cells are described in EP 238 023 and Yelton et al., 1984, Proceedings of the National Academy of Sciences USA 81: 1470-1474. Suitable methods for transforming Fusarium species are described by Malardier et al., 1989, Gene 78: 147-156, and WO 96/00787. Yeast may be transformed using the procedures described by Becker and Guarente, In Abelson, J. N. and Simon, M. I., editors, Guide to Yeast Genetics and Molecular Biology, Methods in Enzymology, Volume 194, pp 182-187, Academic Press, Inc., New York; Ito et al., 1983, Journal of Bacteriology 153: 163; and Hinnen et al., 1978, Proceedings of the National Academy of Sciences USA 75: 1920.

Accordingly, in a preferred embodiment, the host cell is transformed; preferably the host cell is transformed with a polynucleotide construct comprising at least one polynucleotide encoding the one or more enzyme of interest.

### Polynucleotide Constructs

The term “nucleic acid construct” as used herein refers to a nucleic acid molecule, either single- or double-stranded, which is isolated from a naturally occurring gene or which is modified to contain segments of nucleic acids in a manner that would not otherwise exist in nature or which is synthetic. The term nucleic acid construct is synonymous with the term “expression cassette” when the nucleic acid construct contains the control sequences required for expression of a coding sequence of the present invention.

The term “control sequences” is defined herein to include all components necessary for the expression of a polynucleotide encoding a polypeptide of the present invention. Each control sequence may be native or foreign to the nucleotide sequence encoding the polypeptide or native or foreign to each other. Such control sequences include, but are not limited to, a leader, polyadenylation sequence, propeptide sequence, promoter, signal peptide sequence, and transcription terminator. At a minimum, the control sequences include a promoter, and transcriptional and translational stop signals. The control sequences may be provided with linkers for the purpose of introducing specific restriction sites facilitating ligation of the control sequences with the coding region of the nucleotide sequence encoding a polypeptide.

The term “operably linked” denotes herein a configuration in which a control sequence is placed at an appropriate position relative to the coding sequence of the polynucleotide sequence such that the control sequence directs the expression of the coding sequence of a polypeptide.

The term “expression” includes any step involved in the production of the polypeptide including, but not limited to, transcription, post-transcriptional modification, translation, post-translational modification, and secretion.

The term “expression vector” is defined herein as a linear or circular DNA molecule that comprises a polynucleotide encoding a polypeptide of the present invention and is operably linked to additional nucleotides that provide for its expression.

The term “host cell”, as used herein, includes any cell type that is susceptible to transformation, transfection, transduction, and the like with a nucleic acid construct or expression vector comprising a polynucleotide of the present invention.

The term “modification” means herein any chemical modification of the active enzyme polypeptide or a homologous sequence thereof; as well as genetic manipulation of the DNA encoding such a polypeptide. The modification can be a substitution, a deletion and/or an insertion of one or more (several) amino acids as well as replacements of one or more (several) amino acid side chains. Preferably, amino acid changes are of a minor nature, that is conservative amino acid substitutions or insertions that do not significantly affect the folding and/or activity of the protein; small deletions, typically of one to about 30 amino acids; small amino- or carboxyl-terminal extensions, such as an amino-terminal methionine residue; a small linker peptide of up to about 20-25 residues; or a small extension that facilitates purification by changing net charge or another function, such as a poly-histidine tract, an antigenic epitope or a binding domain. Examples of conservative substitutions are within the group of basic amino acids (arginine, lysine and histidine), acidic amino acids (glutamic acid and aspartic acid), polar amino acids (glutamine and asparagine), hydrophobic amino acids (leucine, isoleucine and valine), aromatic amino acids (phenylalanine, tryptophan and tyrosine), and small amino acids (glycine, alanine, serine, threonine and methionine). Amino acid substitutions that do not generally alter specific activity are known in the art and are described, for example, by H. Neurath and R. L. Hill, 1979, In, The Proteins, Academic Press, New York. The most commonly occurring exchanges are Ala/Ser, Val/Ile, Asp/Glu, Thr/Ser, Ala/Gly, Ala/Thr, Ser/Asn, Ala/Val, Ser/Gly, Tyr/Phe, Ala/Pro, Lys/Arg, Asp/Asn, Leu/Ile, Leu/Val, Ala/Glu, and Asp/Gly. In addition to the 20 standard amino acids, non-standard amino acids (such as 4-hydroxyproline, 6-N-methyl lysine, 2-aminoisobutyric acid, isovaline, and alpha-methyl serine) may be substituted for amino acid residues of a wild-type polypeptide. A limited number of non-conservative amino acids, amino acids that are not encoded by the genetic code, and unnatural amino acids may be substituted for amino acid residues. “Unnatural amino acids” have been modified after protein synthesis, and/or have a chemical structure in their side chain(s) different from that of the standard amino acids. Unnatural amino acids can be chemically synthesized, and preferably, are commercially available, and include pipecolic acid, thiazolidine carboxylic acid, dehydroproline, 3- and 4-methylproline, and 3,3-dimethylproline. Alternatively, the amino acid changes are of such a nature that the physico-chemical properties of the polypeptides are altered. For example, amino acid changes may improve the thermal stability of the polypeptide, alter the substrate specificity, change the pH optimum, and the like. Essential amino acids in the parent polypeptide can be identified according to procedures known in the art, such as site-directed mutagenesis or alanine-scanning mutagenesis (Cunningham and Wells, 1989, Science 244: 1081-1085). In the latter technique, single alanine mutations are introduced at every residue in the molecule, and the resultant mutant molecules are tested for biological activity (i.e., enzyme activity) to identify amino acid residues that are critical to the activity of the molecule. See also, Hilton et al., 1996, J. Biol. Chem. 271: 4699-4708. The active site of the enzyme or other biological interaction can also be determined by physical analysis of structure, as determined by such techniques as nuclear magnetic resonance, crystallography, electron diffraction, or photoaffinity labeling, in conjunction with mutation of putative contact site amino acids. See, for example, de Vos et al., 1992, Science 255: 306-312; Smith et al., 1992, J. Mol. Biol. 224: 899-904; Wlodaver et al., 1992, FEBS Lett. 309: 59-64. The identities of essential amino acids can also be inferred from analysis of identities with polypeptides that are related to a polypeptide according to the invention. Single or multiple amino acid substitutions, deletions, and/or insertions can be made and tested using known methods of mutagenesis, recombination, and/or shuffling, followed by a relevant screening procedure, such as those disclosed by Reidhaar-Olson and Sauer, 1988, Science 241: 53-57; Bowie and Sauer, 1989, Proc. Natl. Acad. Sci. USA 86: 2152-2156; WO 95/17413; or WO 95/22625. Other methods that can be used include error-prone PCR, phage display (e.g., Lowman et al., 1991, Biochem. 30: 10832-10837; U.S. Pat. No. 5,223,409; WO 92/06204), and region-directed mutagenesis (Derbyshire et al., 1986, Gene 46: 145; Ner et al., 1988, DNA 7: 127). Mutagenesis/shuffling methods can be combined with high-throughput, automated screening methods to detect activity of cloned, mutagenized polypeptides expressed by host cells (Ness et al., 1999, Nature Biotechnology 17: 893-896). Mutagenized DNA molecules that encode active polypeptides can be recovered from the host cells and rapidly sequenced using standard methods in the art. These methods allow the rapid determination of the importance of individual amino acid residues in a polypeptide of interest, and can be applied to polypeptides of unknown structure.

The present invention also relates to nucleic acid constructs comprising an isolated polynucleotide encoding the active enzyme of the present invention operably linked to one or more (several) control sequences that direct the expression of the coding sequence in a suitable host cell under conditions compatible with the control sequences.

An isolated polynucleotide encoding a polypeptide of the present invention may be manipulated in a variety of ways to provide for expression of the polypeptide. Manipulation of the polynucleotide's sequence prior to its insertion into a vector may be desirable or necessary depending on the expression vector. The techniques for modifying polynucleotide sequences utilizing recombinant DNA methods are well known in the art.

The techniques used to isolate or clone a polynucleotide encoding a polypeptide are known in the art and include isolation from genomic DNA, preparation from cDNA, or a combination thereof. The cloning of the polynucleotides of the present invention from such genomic DNA can be effected, e.g., by using the well known polymerase chain reaction (PCR) or antibody screening of expression libraries to detect cloned DNA fragments with shared structural features. See, e.g., Innis et al., 1990, PCR: A Guide to Methods and Application, Academic Press, New York. Other nucleic acid amplification procedures such as ligase chain reaction (LCR), ligated activated transcription (LAT) and nucleotide sequence-based amplification (NASBA) may be used. The polynucleotides may be cloned from one of the abovelisted strains, or another or related organism and thus, for example, may be an allelic or species variant of the polypeptide encoding region of the nucleotide sequence.

The control sequence may be an appropriate promoter sequence, a nucleotide sequence that is recognized by a host cell for expression of a polynucleotide encoding a polypeptide of the present invention. The promoter sequence contains transcriptional control sequences that mediate the expression of the polypeptide. The promoter may be any nucleotide sequence that shows transcriptional activity in the host cell of choice including mutant, truncated, and hybrid promoters, and may be obtained from genes encoding extracellular or intracellular polypeptides either homologous or heterologous to the host cell.

Examples of suitable promoters for directing the transcription of the nucleic acid constructs of the present invention, especially in a bacterial host cell, are the promoters obtained from the E. coli lac operon, Streptomyces coelicolor agarase gene (dagA), Bacillus subtilis levansucrase gene (sacB), Bacillus licheniformis alpha-amylase gene (amyL), Bacillus stearothermophilus maltogenic amylase gene (amyM), Bacillus amyloliquefaciens alpha-amylase gene (amyQ), Bacillus licheniformis penicillinase gene (penP), Bacillus subtilis xylA and xylB genes, and prokaryotic beta-lactamase gene (Villa-Kamaroff et al., 1978, Proceedings of the National Academy of Sciences USA 75: 3727-3731), as well as the tac promoter (DeBoer et al., 1983, Proceedings of the National Academy of Sciences USA 80: 21-25). Further promoters are described in “Useful proteins from recombinant bacteria” in Scientific American, 1980, 242: 74-94; and in Sambrook et al., 1989, supra.

Examples of suitable promoters for directing the transcription of the nucleic acid constructs of the present invention in a filamentous fungal host cell are promoters obtained from the genes for Aspergillus oryzae TAKA amylase, Rhizomucor miehei aspartic proteinase, Aspergillus niger neutral alpha-amylase, Aspergillus niger acid stable alpha-amylase, Aspergillus niger or Aspergillus awamori glucoamylase (glaA), Rhizomucor miehei lipase, Aspergillus oryzae alkaline protease, Aspergillus oryzae triose phosphate isomerase, Aspergillus nidulans acetamidase, Fusarium venenatum amyloglucosidase (WO 00/56900), Fusarium venenatum Daria (WO 00/56900), Fusarium venenatum Quinn (WO 00/56900), Fusarium oxysporum trypsin-like protease (WO 96/00787), Trichoderma reesei beta-glucosidase, Trichoderma reesei cellobiohydrolase I, Trichoderma reesei cellobiohydrolase II, Trichoderma reesei endoglucanase I, Trichoderma reesei endoglucanase II, Trichoderma reesei endoglucanase III, Trichoderma reesei endoglucanase IV, Trichoderma reesei endoglucanase V, Trichoderma reesei xylanase I, Trichoderma reesei xylanase II, Trichoderma reesei beta-xylosidase, as well as the NA2-tpi promoter (a hybrid of the promoters from the genes for Aspergillus niger neutral alpha-amylase and Aspergillus oryzae triose phosphate isomerase); and mutant, truncated, and hybrid promoters thereof.

In a yeast host, useful promoters are obtained from the genes for Saccharomyces cerevisiae enolase (ENO-1), Saccharomyces cerevisiae galactokinase (GAL1), Saccharomyces cerevisiae alcohol dehydrogenase/glyceraldehyde-3-phosphate dehydrogenase (ADH1, ADH2/GAP), Saccharomyces cerevisiae triose phosphate isomerase (TPI), Saccharomyces cerevisiae metallothionein (CUP1), and Saccharomyces cerevisiae 3-phosphoglycerate kinase. Other useful promoters for yeast host cells are described by Romanos et al., 1992, Yeast 8: 423-48

The control sequence may also be a suitable transcription terminator sequence, a sequence recognized by a host cell to terminate transcription. The terminator sequence is operably linked to the 3′ terminus of the nucleotide sequence encoding the polypeptide. Any terminator that is functional in the host cell of choice may be used in the present invention.

Preferred terminators for filamentous fungal host cells are obtained from the genes for Aspergillus oryzae TAKA amylase, Aspergillus niger glucoamylase, Aspergillus nidulans anthranilate synthase, Aspergillus niger alpha-glucosidase, and Fusarium oxysporum trypsin-like protease.

Preferred terminators for yeast host cells are obtained from the genes for Saccharomyces cerevisiae enolase, Saccharomyces cerevisiae cytochrome C(CYC1), and Saccharomyces cerevisiae glyceraldehyde-3-phosphate dehydrogenase. Other useful terminators for yeast host cells are described by Romanos et al., 1992, supra.

The control sequence may also be a suitable leader sequence, a nontranslated region of an mRNA that is important for translation by the host cell. The leader sequence is operably linked to the 5′ terminus of the nucleotide sequence encoding the polypeptide. Any leader sequence that is functional in the host cell of choice may be used in the present invention.

Preferred leaders for filamentous fungal host cells are obtained from the genes for Aspergillus oryzae TAKA amylase and Aspergillus nidulans triose phosphate isomerase.

Suitable leaders for yeast host cells are obtained from the genes for Saccharomyces cerevisiae enolase (ENO-1), Saccharomyces cerevisiae 3-phosphoglycerate kinase, Saccharomyces cerevisiae alpha-factor, and Saccharomyces cerevisiae alcohol dehydrogenase/glyceraldehyde-3-phosphate dehydrogenase (ADH2/GAP).

The control sequence may also be a polyadenylation sequence, a sequence operably linked to the 3′ terminus of the nucleotide sequence and, when transcribed, is recognized by the host cell as a signal to add polyadenosine residues to transcribed mRNA. Any polyadenylation sequence that is functional in the host cell of choice may be used in the present invention.

Preferred polyadenylation sequences for filamentous fungal host cells are obtained from the genes for Aspergillus oryzae TAKA amylase, Aspergillus niger glucoamylase, Aspergillus nidulans anthranilate synthase, Fusarium oxysporum trypsin-like protease, and Aspergillus niger alpha-glucosidase.

Useful polyadenylation sequences for yeast host cells are described by Guo and Sherman, 1995, Molecular Cellular Biology 15: 5983-5990.

The control sequence may also be a signal peptide coding sequence that codes for an amino acid sequence linked to the amino terminus of a polypeptide and directs the encoded polypeptide into the cell's secretory pathway. The 5′ end of the coding sequence of the nucleotide sequence may inherently contain a signal peptide coding sequence naturally linked in translation reading frame with the segment of the coding sequence that encodes the secreted polypeptide. Alternatively, the 5′ end of the coding sequence may contain a signal peptide coding sequence that is foreign to the coding sequence. The foreign signal peptide coding sequence may be required where the coding sequence does not naturally contain a signal peptide coding sequence. Alternatively, the foreign signal peptide coding sequence may simply replace the natural signal peptide coding sequence in order to enhance secretion of the polypeptide. However, any signal peptide coding sequence that directs the expressed polypeptide into the secretory pathway of a host cell of choice, i.e., secreted into a culture medium, may be used in the present invention.

Effective signal peptide coding sequences for bacterial host cells are the signal peptide coding sequences obtained from the genes for Bacillus NCIB 11837 maltogenic amylase, Bacillus stearothermophilus alpha-amylase, Bacillus licheniformis subtilisin, Bacillus licheniformis beta-lactamase, Bacillus stearothermophilus neutral proteases (nprT, nprS, nprM), Bacillus clausii alcaline protease (aprH) and Bacillus subtilis prsA. Further signal peptides are described by Simonen and Palva, 1993, Microbiological Reviews 57: 109-137.

Effective signal peptide coding sequences for filamentous fungal host cells are the signal peptide coding sequences obtained from the genes for Aspergillus oryzae TAKA amylase, Aspergillus niger neutral amylase, Aspergillus niger glucoamylase, Rhizomucor miehei aspartic proteinase, Humicola insolens cellulase, Humicola insolens endoglucanase V, and Humicola lanuginosa lipase.

Useful signal peptides for yeast host cells are obtained from the genes for Saccharomyces cerevisiae alpha-factor and Saccharomyces cerevisiae invertase. Other useful signal peptide coding sequences are described by Romanos et al., 1992, supra.

The control sequence may also be a propeptide coding sequence that codes for an amino acid sequence positioned at the amino terminus of a polypeptide. The resultant polypeptide is known as a proenzyme or propolypeptide (or a zymogen in some cases). A propeptide is generally inactive and can be converted to a mature active polypeptide by catalytic or autocatalytic cleavage of the propeptide from the propolypeptide. The propeptide coding sequence may be obtained from the genes for Bacillus subtilis alkaline protease (aprE), Bacillus subtilis neutral protease (nprT), Saccharomyces cerevisiae alpha-factor, Rhizomucor miehei aspartic proteinase, and Myceliophthora thermophila laccase (WO 95/33836).

Where both signal peptide and propeptide sequences are present at the amino terminus of a polypeptide, the propeptide sequence is positioned next to the amino terminus of a polypeptide and the signal peptide sequence is positioned next to the amino terminus of the propeptide sequence.

It may also be desirable to add regulatory sequences that allow the regulation of the expression of the polypeptide relative to the growth of the host cell. Examples of regulatory systems are those that cause the expression of the gene to be turned on or off in response to a chemical or physical stimulus, including the presence of a regulatory compound. Regulatory systems in prokaryotic systems include the lac, tac, xyl and trp operator systems. In yeast, the ADH2 system or GAL1 system may be used. In filamentous fungi, the TAKA alpha-amylase promoter, Aspergillus niger glucoamylase promoter, and Aspergillus oryzae glucoamylase promoter may be used as regulatory sequences. Other examples of regulatory sequences are those that allow for gene amplification. In eukaryotic systems, these regulatory sequences include the dihydrofolate reductase gene that is amplified in the presence of methotrexate, and the metallothionein genes that are amplified with heavy metals. In these cases, the nucleotide sequence encoding the polypeptide would be operably linked with the regulatory sequence.

### Expression Vectors

The present invention also relates to recombinant expression vectors comprising a polynucleotide of the present invention, a promoter, and transcriptional and translational stop signals. The various nucleic acids and control sequences described herein may be joined together to produce a recombinant expression vector that may include one or more (several) convenient restriction sites to allow for insertion or substitution of the nucleotide sequence encoding the polypeptide at such sites. Alternatively, a polynucleotide sequence of the present invention may be expressed by inserting the nucleotide sequence or a nucleic acid construct comprising the sequence into an appropriate vector for expression. In creating the expression vector, the coding sequence is located in the vector so that the coding sequence is operably linked with the appropriate control sequences for expression.

The recombinant expression vector may be any vector (e.g., a plasmid or virus) that can be conveniently subjected to recombinant DNA procedures and can bring about expression of the nucleotide sequence. The choice of the vector will typically depend on the compatibility of the vector with the host cell into which the vector is to be introduced. The vectors may be linear or closed circular plasmids.

The vector may be an autonomously replicating vector, i.e., a vector that exists as an extrachromosomal entity, the replication of which is independent of chromosomal replication, e.g., a plasmid, an extrachromosomal element, a minichromosome, or an artificial chromosome. The vector may contain any means for assuring self-replication. Alternatively, the vector may be one that, when introduced into the host cell, is integrated into the genome and replicated together with the chromosome(s) into which it has been integrated. Furthermore, a single vector or plasmid or two or more vectors or plasmids that together contain the total DNA to be introduced into the genome of the host cell, or a transposon, may be used.

The vectors of the present invention preferably contain one or more (several) selectable markers that permit easy selection of transformed, transfected, transduced, or the like cells. A selectable marker is a gene the product of which provides for biocide or viral resistance, resistance to heavy metals, prototrophy to auxotrophs, and the like. Examples of bacterial selectable markers are the dal genes from Bacillus subtilis or Bacillus licheniformis, or markers that confer antibiotic resistance such as ampicillin, kanamycin, chloramphenicol, or tetracycline resistance. Suitable markers for yeast host cells are ADE2, HIS3, LEU2, LYS2, MET3, TRP1, and URA3. Selectable markers for use in a filamentous fungal host cell include, but are not limited to, amdS (acetamidase), argB (ornithine carbamoyltransferase), bar (phosphinothricin acetyltransferase), hph (hygromycin phosphotransferase), niaD (nitrate reductase), pyrG (orotidine-5′-phosphate decarboxylase), sC (sulfate adenyltransferase), and trpC (anthranilate synthase), as well as equivalents thereof. Preferred for use in an Aspergillus cell are the amdS and pyrG genes of Aspergillus nidulans or Aspergillus oryzae and the bar gene of Streptomyces hygroscopicus.

The vectors of the present invention preferably contain an element(s) that permits integration of the vector into the host cell's genome or autonomous replication of the vector in the cell independent of the genome.

For integration into the host cell genome, the vector may rely on the polynucleotide's sequence encoding the polypeptide or any other element of the vector for integration into the genome by homologous or nonhomologous recombination. Alternatively, the vector may contain additional nucleotide sequences for directing integration by homologous recombination into the genome of the host cell at a precise location(s) in the chromosome(s). To increase the likelihood of integration at a precise location, the integrational elements should preferably contain a sufficient number of nucleic acids, such as 100 to 10,000 base pairs, preferably 400 to 10,000 base pairs, and most preferably 800 to 10,000 base pairs, which have a high degree of identity to the corresponding target sequence to enhance the probability of homologous recombination. The integrational elements may be any sequence that is homologous with the target sequence in the genome of the host cell. Furthermore, the integrational elements may be non-encoding or encoding nucleotide sequences. On the other hand, the vector may be integrated into the genome of the host cell by non-homologous recombination.

For autonomous replication, the vector may further comprise an origin of replication enabling the vector to replicate autonomously in the host cell in question. The origin of replication may be any plasmid replicator mediating autonomous replication that functions in a cell. The term “origin of replication” or “plasmid replicator” is defined herein as a nucleotide sequence that enables a plasmid or vector to replicate in vivo.

Examples of bacterial origins of replication are the origins of replication of plasmids pBR322, pUC19, pACYC177, and pACYC184 permitting replication in E. coli, and pUB110, pE194, pTA1060, and pAMβ1 permitting replication in Bacillus.

Examples of origins of replication for use in a yeast host cell are the 2 micron origin of replication, ARS1, ARS4, the combination of ARS1 and CEN3, and the combination of ARS4 and CEN6.

Examples of origins of replication useful in a filamentous fungal cell are AMA1 and ANSI (Gems et al., 1991, Gene 98: 61-67; Cullen et al., 1987, Nucleic Acids Research 15: 9163-9175; WO 00/24883). Isolation of the AMA1 gene and construction of plasmids or vectors comprising the gene can be accomplished according to the methods disclosed in WO 00/24883.

More than one copy of a polynucleotide of the present invention may be inserted into a host cell to increase production of the gene product. An increase in the copy number of the polynucleotide can be obtained by integrating at least one additional copy of the sequence into the host cell genome or by including an amplifiable selectable marker gene with the polynucleotide where cells containing amplified copies of the selectable marker gene, and thereby additional copies of the polynucleotide, can be selected for by cultivating the cells in the presence of the appropriate selectable agent.

The procedures used to ligate the elements described above to construct the recombinant expression vectors of the present invention are well known to one skilled in the art (see, e.g., Sambrook et al., 1989, supra).

### Synthase Inhibitors

In a preferred embodiment of the first aspect of the invention, the one or more synthase inhibitor comprises an inhibitor which inhibits the synthesis of at least one amino acid and/or at least one fatty acid; preferably the one or more synthase inhibitor comprises 5-nitro-2-benzimidazolinone, glyphosate, carboxymethoxylhydroxylamine, carboxymethoxylamine, O-allylhydroxylamine, indole acrylic acid, O-(carboxymethyl) hydroxylamine hemihydrochloride, an imidazolinone, or sulfonyl urea; more preferably the one or more synthase inhibitor comprises an inhibitor which inhibits the synthesis of methionine, preferably the one or more synthase inhibitor comprises carboxymethoxylhydroxylamine; and still more preferably the one or more synthase inhibitor comprises triclosan, cerulenin, thiolactomycin or diazaborin.

In another preferred embodiment of the first aspect of the invention, the one or more synthase inhibitor comprises an inhibitor which inhibits glucose-6-phophatase; preferably the one or more synthase inhibitor comprises phloretin, pyridoxal phosphate, theilavin A, 2-hydroxy-5-nitrobenzaldehyde or Mumbaistatin.

In yet another preferred embodiment of the first aspect of the invention, the one or more synthase inhibitor comprises an inhibitor which inhibits glucosamine-6P synthase; preferably the one or more synthase inhibitor comprises amitrole or aptamine.

Another preferred embodiment relates to a method of the first aspect of the invention, wherein the one or more synthase inhibitor comprises an inhibitor which inhibits fructose 1,6 biphosphatase; preferably the one or more synthase inhibitor comprises 2,3-dihydro-1H-cyclopenta[b]quinoline.

### EXAMPLES

### Example 1

### Selection of Transformants

The purpose of this example is to demonstrate selection of transformants, which express an active enzyme of interest under particular growth conditions; the transformants which do not express active enzyme under these conditions cannot grow. The synthesis of at least one essential component(s) in the cell is inhibited by one or more synthesis inhibitor added to the growth medium, so the essential component(s) can only be obtained from the medium by the host cell if the active enzyme of interest is being produced.

Construction of Plasmid pENI3659

Plasmid pENI3659 is a further development of pENI3420. Plasmid pENI3420 contains a neutral amylase 2 promoter from Aspergillus, which gives rise to gene expression in yeast. The plasmid is a pYES2.0 derivative, which replicates in yeast and can be selected by ura3 selection.

A PCR was made using pENI3420 as template and oligo 180804L1 (10 microM), 180804L2 (0.1 microM), and 180804L3 (10 microM) using PWO polymerase as recommended by manufacturer (Roche).

180804L1:

(SEQ ID NO: 1)

aagggatcctcgaggtaccacgcgtgaattcactagtgcatgcaagctt
180804L2:

(SEQ ID NO: 2)

gtcaccctctagatctcgacttaattaagcttgcatgcactagtgaat
180804L3:

(SEQ ID NO: 3)

gttccggttacctttgcggataag

Plasmid pENI3420 and the generated PCR fragment were both cut with the restriction enzymes BamHI and BstEII.

The cut vector pENI3420 and the cut PCR fragment were purified from agarose gel, ligated and transformed into the E. coli strain (Sambrook and Russell: Molecular cloning—a laboratory manual 2001 Cold spring Harbor laboratory press NY). Plasmid preparations were made and sequenced.

The correct clone contained a multilinker site infront of the promoter, thus making it suitable for cloning of desired genes.

Construction of Plasmid pENI2419

Plasmid pENI2419 was constructed as shown in WO 97/04079 A1 (Novozymes A/S) using plasmid pAHL disclosed in WO 92/05249 A1 (Novozymes A/S) as template and oligo21350.

Oligo21350:

(SEQ ID NO: 4)

gaacccttgtccccgtccggcgacgagacatcgtgaagatagaaggc

Construction of Plasmid pENI3791

Plasmid pENI2419 was cut with BamHI/XhoI and the fragment containing the lipase gene was cloned into plasmid pENI3659 cut with BamHI/XhoI, thus creating pENI3791.

Construction of Plasmid pENI4158

Two PCR reactions were run using PWO polymerase:

Template: pENI2419. Oligo 170904L1 and oligo 190804L1

Template: pENI3420. Oligo 190804L2 and 0603002j1

The two PCR fragments were isolated and purified from agarose gel, and used in a third PCR reaction using PWO polymerase:

Template: The PCR fragments from the PCR reations a) and b), together with oligos 170904L1 and 060302j1.

| | |
|---|---|
| 170904L1: | |
| ccatttcactactattatgc | (SEQ ID NO: 5) |
| 190804L1: | |
| ctcggggaggtctcgcaggatctgtt | (SEQ ID NO: 6) |
| 190804L2: | |
| gcgagacctccccgagggccagcttcccca | (SEQ ID NO: 7) |
| 060302j1: | |
| agagcttaaagtatgtcccttg | (SEQ ID NO: 8) |

The PCR fragments produced in the third reaction and plasmid pENI3791 were cut with BamHI and MfeI, isolated and purified from agarose gel, and ligated, thus creating pENI4158.

### Example 2

### Cerulinine Inhibition of Fatty Acid Synthesis in Yeast

The purpose of this example is to show that cerulinine inhibits yeast growth due to inhibition of fatty acid synthase and to show that expression and secretion of lipase in the presence of a triglyceride can restore yeast growth in the presence of cerulinine, due to the free fatty acids generated by the lipase, when hydrolysing triglyceride.

Plasmids pENI3659 (control) and pENi4158 (with lipase gene) were transformed into the yeast strain Saccharomyces cerevisiae JG 169 (MAT-alpha; ura 3-52; leu 2-3, 112; his 3-D200; pep 4-113; prc1::HIS3; prb1::LEU2). The transformed yeast was streaked onto plates containing cerulenine and olive oil.

Only yeast cells transformed with pENI4158, which express active lipase, were able to grow on the triglyceride plates in the presence of cerulinine.

### Triglyceride Plates:

To 450 ml SC-ura-basic agar was added 50 ml 20% glucose and 10 ml olive oil. An UltraTurax™ ultrasound generator (IKA Labortechnik, Germany) was used to disperse the olive oil. 5 mg cerulenin dissolve in 1 ml ethanol was added and 500 microliter ampicillin (100 mg/ml)). This mix was poured into four 9 cm petri dishes.

SC-ura-basic agar: 7.5 g Yeast Nitrogen Base without amino acids, 11.3 g Bernstein, 6.8 g NaOH, 5.6 g casamino acids, 0.1 g L-tryptophan, 20 g agar in total of 900 ml water.

### Example 3

### Selection of Shuffled Lipase

Four different wildtype genes encoding secreted and active lipases having an overall amino acid sequence identity of 32% (when comparing all four lipase genes) were shuffled: The gene encoding one lipase from pENI2419 (Humicola lanuginosa; example 1) is shown in SEQ ID NO:9, and the encoded amino acid sequence is shown in SEQ ID NO:10.

The gene encoding the second lipase (ND002444 Nectria sp) is shown in SEQ ID NO:11 and the amino acid sequence in SEQ ID NO:12.

The gene encoding the third lipase (ND002119 Fusarium sp) is shown in SEQ ID NO:13 and the amino acid sequence in SEQ ID NO:14.

The gene encoding the fourth lipase (ND002652 Gibberella zeae) is shown in SEQ ID NO:15 and the amino acid sequence in SEQ ID NO:16.

The following PCR were made using PWO polymerase as recommended by manufacture (Roche), the primersequences are in the sequence listing:

TABLE 1
PCR setup with template and primers.

| PCR fragment | | Fwd primer | Rev primer |
|---|---|---|---|
| number | Template | (SEQ ID NO: #) | (SEQ ID NO: #) |
| 1 | ND002444 | 060302j1 (8) | 220506L1rev (17) |
| 2 | ND002444 | 220506L1fwp (18) | 220506L2rev (19) |
| 3 | ND002444 | 220506L2fwp (20) | 220506L3rev (21) |
| 4 | ND002444 | 220506L3fwp (22) | 220506L4rev (23) |
| 5 | ND002444 | 220506L4fwp (24) | 220506L5rev (25) |
| 6 | ND002444 | 220506L5fwp (26) | 220506L6rev (27) |
| 7 | ND002444 | 220506L6fwp (28) | 230506L1 (29) |
| 8 | ND002119 | 060302j1 (8) | 220506L7rev (30) |
| 9 | ND002119 | 220506L7fwp (31) | 220506L8rev (32) |
| 10 | ND002119 | 220506L8fwp (33) | 220506L9rev (34) |
| 11 | ND002119 | 220506L9fwp (35) | 220506L10rev (36) |
| 12 | ND002119 | 220506L10fwp (37) | 220506L11rev (38) |
| 13 | ND002119 | 220506L11fwp (39) | 220506L12rev (40) |
| 14 | ND002119 | 220506L12fwp (41) | 230506L1 (29) |
| 15 | pENI2419 | 060302j1 (8) | 220506L13rev (42) |
| 16 | pENI2419 | 220506L13fwp (43) | 220506L14rev (44) |
| 17 | pENI2419 | 220506L14fwp (45) | 220506L15rev (46) |
| 18 | pENI2419 | 220506L15fwp (47) | 220506L16rev (48) |
| 19 | pENI2419 | 220506L16fwp (49) | 220506L17rev (50) |
| 20 | pENI2419 | 220506L17fwp (51) | 220506L18rev (52) |
| 21 | pENI2419 | 220506L18fwp (53) | 230506L1 (29) |
| 22 | ND002652 | 230506L2 (54) | 220506L19rev (55) |
| 23 | ND002652 | 220506L19fwp (56) | 220506L20rev (57) |
| 24 | ND002652 | 220506L20fwp (58) | 220506L21rev (59) |
| 25 | ND002652 | 220506L21fwp (60) | 220506L22rev (61) |
| 26 | ND002652 | 220506L22fwp (62) | 220506L23rev (63) |
| 27 | ND002652 | 220506L23fwp (64) | 220506L24rev (65) |
| 28 | ND002652 | 220506L24fwp (66) | 230506L3 (67) |

The following PCR was made using PWO polymerase as recommended by the manufacturer (Roche) in a total volume of 100 microliter:

10 microliter of PCR fragment no's: 1, 7, 8, 14, 15, 21, 22, 28.
1 microliter of PCR fragments no's: 2, 3, 4, 5, 6, 9, 10, 11, 12, 13, 16, 17, 18, 19, 20, 23, 24, 25, 26, 27.

The PCR fragment was cut with KpnI and SpeI and cloned into pENI3659 cut with the same enzymes. Approx. 30.000 E. coli clones were obtained.

DNA prep was made from these clones and transformed into yeast (ATCC 26787 (MATa,SUC2,mal,gal2,CUP1) selected to be Ura-minus on 5-FOA plates) obtaining approx. 120,000 yeast transformants.

The library was plated onto triglyceride plates (see example 2). A number of yeast transformants grew on these plates.

Plasmid DNA was isolated from the yeast transformants using Bio101-systems (FastDNA spinkit cat. 6560-200) as recommended by the manufacturer. The DNA prep was transformed into E. coli, and DNA was prepared from the E. coli transformants. The selected shuffled lipases were identified by sequencing of the plasmids.

### Example 4

### Plate-Selection of Protease-Secreting Bacillus

In this example transformants are selected, which can express an active protease enzyme under the given growth conditions. The selection mechanism is based on that synthesis of one or more essential component in the transformant cell is inhibited by the presence of a synthesis inhibitor(s) in the growth medium. The essential component(s) can only be obtained if a protein of interest is produced by the transformant which renders it able to grow in the presence of the inhibitor.

We wanted to find a concentration of carboxymethoxylhydroxylamine (CMA) that allows only protease-secreting Bacillus colonies to grow in the presence of the small peptide met-gly-met-met (MGMM). CMA inhibits the methionine synthesis in Bacillus cells, which severely hampers their growth. However, in the presence of MGMM, protease-secreting Bacillus cells can grow on CMA containing media. Secreted proteases degrade the MGMM peptide thereby releasing free methionine residues, which are taken up by the protese-secreting Bacillus cells and used for protein synthesis.

### Experiment Description:

500 ml LB agar was melted and placed in an incubator until the agar reached 60° C. 5 ml met-gly-met-met (MGMM, Sigma M4786-250MG) (5 mg/ml) and 300 microliter 1% chloramphenicol (in 70% ethanol) was added to the agar.

4×25 ml media was transferred with a 25 ml Stripette™ to clean 25 ml Nunc™ containers. Different amounts of CMA (Aldrich C13408-1 g) were added to the media as indicated in the scheme below. All 25 ml media was poured into 9 cm petri dishes and dried in a clean bench; except for the “6×CMA on the plate” where 25 ml media was poured on a 9 cm petridish and dried and 600 microliter CMA (0.56 mg/ml) was spread on the plate afterwards.

TABLE 2

| | 0.56 mg/ml | The mg/L conc. |
|---|---|---|
| Name | CMA | CMA in the plate |
| Control without CMA | 0 | 0 |
| 5xCMA in the plate | 500 | 11.2 |
| 6xCMA in the plate | 600 | 13.4 |
| 6xCMA on the plate | 600 | 13.4 |
| 7xCMA in the plate | 700 | 15.7 |
| 8xCMA in the plate | 800 | 17.9 |

A Bacillus strain secreting a wildtype protease denoted ‘10R’ (WO 2004/111220) and a Bacillus strain secreting inactive 10R protease (comprising the substitution S143A in the 10R amino acid sequence) were both inoculated in 200 microliter LB-bouillon from freeze-stock. The two micro-organisms were streaked on the 6 different types of plates. All plates were incubated upside down at 37° C. overnight. The results are shown in table 3 below.

TABLE 3

| | Growth on the plate | |
|---|---|---|
| Name | 10R WT (SOL000) | 10R inactive (SOL218) |
| Control without CMA | Yes | Yes |
| | | (a little smaller than WT) |
| 5xCMA in the plate | Yes | No |
| | (smaller than control) | |
| 6xCMA in the plate | No | No |
| 6xCMA on the plate | No | No |
| 7xCMA in the plate | No | No |
| 8xCMA in the plate | No | No |

It is clear from table 3 that LB agar with 11.2 mg/L CMA as well as MGMM in the plate can be used to select only those Bacillus transformant cells, which secrete active 10R protease, since only those can grow on the plates under the given conditions.

1-14. (canceled)

15. A method for selecting at least one Gram positive host cell secreting one or more active enzyme of interest, said method comprising the steps of:
a) providing a growth medium comprising one or more synthase inhibitor, which inhibits the synthesis of at least one essential compound in the host cell, and further comprising one or more component, which in the presence of the one or more active enzyme of interest is converted into the at least one essential compound, thereby allowing the host cell to grow;
b) cultivating the host cell in or on the growth medium of step (a); and
c) selecting at least one host cell capable of growing in or on the growth medium of step (a), which host cell secretes one or more active enzyme of interest.

16. The method of claim 15, wherein the host cell is transformed.

17. The method of claim 15, wherein the host cell is transformed with a polynucleotide construct comprising at least one polynucleotide encoding the one or more enzyme of interest.

18. The method of claim 15, wherein the host cell is a Bacillus cell.

19. The method of claim 15, wherein the host cell is a Bacillus alkalophilus, Bacillus amyloliquefaciens, Bacillus brevis, Bacillus circulans, Bacillus clausii, Bacillus coagulans, Bacillus firmus, Bacillus lautus, Bacillus lentus, Bacillus licheniformis, Bacillus megaterium, Bacillus pumilus, Bacillus stearothermophilus, Bacillus subtilis or a Bacillus thuringiensis cell.

20. The method of claim 15, wherein the one or more active enzyme of interest is heterologous or homologous.

21. The method of claim 15, wherein the one or more active enzyme of interest comprises an oxidoreductase, a transferase, a hydrolase, a lyase, an isomerase and/or a ligase.

22. The method of claim 15, wherein the one or more active enzyme of interest comprises a lipase and/or a protease.

23. The method of claim 15, wherein the one or more synthase inhibitor comprises an inhibitor which inhibits the synthesis of at least one amino acid and/or at least one fatty acid.

24. The method of claim 23, wherein the one or more synthase inhibitor comprises 5-nitro-2-benzimidazolinone, glyphosate, carboxymethoxylhydroxylamine, carboxymethoxylamine, O-allylhydroxylamine, indole acrylic acid, O-(carboxymethyl) hydroxylamine hemihydrochloride, an imidazolinone or sulfonyl urea.

25. The method of claim 23, wherein the one or more synthase inhibitor comprises an inhibitor which inhibits the synthesis of methionine.

26. The method of claim 23, wherein the one or more synthase inhibitor comprises carboxymethoxylhydroxylamine.

27. The method of claim 23, wherein the one or more synthase inhibitor comprises triclosan, cerulenin, thiolactomycin or diazaborin.

28. The method of claim 15, wherein the one or more synthase inhibitor comprises an inhibitor which inhibits glucose-6-phophatase.

29. The method of claim 15, wherein the one or more synthase inhibitor comprises phloretin, pyridoxal phosphate, theilavin A, 2-hydroxy-5-nitrobenzaldehyde or Mumbaistatin.

30. The method of claim 15, wherein the one or more synthase inhibitor comprises an inhibitor which inhibits glucosamine-6P synthase.

31. The method of claim 15, wherein the one or more synthase inhibitor comprises amitrole or aptamine.

32. The method of claim 15, wherein the one or more synthase inhibitor comprises an inhibitor which inhibits fructose 1,6 biphosphatase.

33. The method of claim 15, wherein the one or more synthase inhibitor comprises 2,3-dihydro-1H-cyclopenta[b]quinoline.


SEQUENCE LISTING (67 sequences)

SEQ ID NO: 1 - DNA, 49 bp, artificial sequence
  Primer 180804L1
aagggatcctcgaggtaccacgcgtgaattcactagtgcatgcaagctt

SEQ ID NO: 2 - DNA, 48 bp, artificial sequence
  Primer 180804L2
gtcaccctctagatctcgacttaattaagcttgcatgcactagtgaat

SEQ ID NO: 3 - DNA, 24 bp, artificial sequence
  Primer 180804L3
gttccggttacctttgcggataag

SEQ ID NO: 4 - DNA, 47 bp, artificial sequence
  Primer Oligo21350
gaacccttgtccccgtccggcgacgagacatcgtgaagatagaaggc

SEQ ID NO: 5 - DNA, 20 bp, artificial sequence
  Primer 170904L1
ccatttcactactattatgc

SEQ ID NO: 6 - DNA, 26 bp, artificial sequence
  Primer 190804L1
ctcggggaggtctcgcaggatctgtt

SEQ ID NO: 7 - DNA, 30 bp, artificial sequence
  Primer 190804L2
gcgagacctccccgagggccagcttcccca

SEQ ID NO: 8 - DNA, 22 bp, artificial sequence
  Primer 060302j1
agagcttaaagtatgtcccttg

SEQ ID NO: 9 - DNA, 873 bp, Humicola lanuginosa
  CDS (1)..(873)
atgaggagctcccttgtgctgttctttgtctctgcgtggacggccttggccagtcctattcgtcgagaggtctcgcaggatctgtttaaccagttcaatctctttgcacagtattctgcagccgcatactgcggaaaaaacaatgatgccccagctggtacaaacattacgtgcacgggaaatgcctgccccgaggtagagaaggcggatgcaacgtttctctactcgtttgaagactctggagtgggcgatgtcaccggcttccttgctctcgacaacacgaacaaattgatcgtcctctctttccgtggctctcgttccatagagaactggatcgggaatcttaacttcgacttgaaagaaataaatgacatttgctccggctgcaggggacatgacggcttcacttcgtcctggaggtctgtagccgatacgttaaggcagaaggtggaggatgctgtgagggagcatcccgactatcgcgtggtgtttaccggacatagcttgggtggtgcattggcaactgttgccggagcagacctgcgtggaaatgggtatgatatcgacgtgttttcatatggcgccccccgagtcggaaacagggcttttgcagaattcctgaccgtacagaccggcggaacactctaccgcattacccacaccaatgatattgtccctagactcccgccgcgcgaattcggttacagccattctagcccagaatactggatcaaatctggaacccttgtccccgtccggcgacgagacatcgtgaagatagaaggcatcgatgccaccggcggcaataaccagcctaacattccggatatccctgcgcacctatggtacttcgggttaattgggacatgtctt

SEQ ID NO: 10 - protein, 291 aa, Humicola lanuginosa
MRSSLVLFFVSAWTALASPIRREVSQDLFNQFNLFAQYSAAAYCGKNNDAPAGTNITCTGNACPEVEKADATFLYSFEDSGVGDVTGFLALDNTNKLIVLSFRGSRSIENWIGNLNFDLKEINDICSGCRGHDGFTSSWRSVADTLRQKVEDAVREHPDYRVVFTGHSLGGALATVAGADLRGNGYDIDVFSYGAPRVGNRAFAEFLTVQTGGTLYRITHTNDIVPRLPPREFGYSHSSPEYWIKSGTLVPVRRRDIVKIEGIDATGGNNQPNIPDIPAHLWYFGLIGTCL

SEQ ID NO: 11 - DNA, 1029 bp, Nectria sp
  CDS (1)..(1029)
atgcgtcttctccctgccctctccgtggtcggcgttgccagcgctgcctccatcaagagctatcttcatgcctttgaggagcgagctgttactgtgacctcccagaacctcgcaaacttcaagttctacgtccagcatgccactgccgcgtactgtaactacgaccgcgcagctggagccttgatttcatgctcgagcaactgcccaagtattgaaagcaatgctgctaagattgtgggatccttcggaggcgaggatacgggcattgcaggctacgtctcaactgacgcaactcgcaaggagattgtcgtctctatccgtggcagtattaacgtccgcaactggatcacaaacctcgacttcgtctggagttcctgctcagatctgtcgagcaactgcaaggcccacgctggcttcaaagatgcttgggatgagatctccaccgctgccaaagctgcagtcgtctcggcgaagaaggccaacccaagctacaccatcgtcgccacgggacactcccttggtggtgctgttgctaccttagcagctgcttacatccgagctgctggatatagtgtcgatctgtacacgttcggctcgccacgtgtaggaaatgactacttcgccaacttcgtcaccagccaagccggagctgaataccgcgtgacacacctcgacgaccctgttcctcgtcttccacccatcctctttggctaccgtcatacgtctcctgagtactggctgtcaaacggaggcgctactacgacgacctatagtctgtcagacatcgtggtatgcgagggtatcgccaacaccgactgcaatgccggcacgcttggccttgatattattgcccacctcatatacttccaggatacttcggcatgcaacaccggattcacgtggaagcgcgacacgttgtcggatgcagagctcgaggagatggtgaacaagtgggctgagcaggatgtcgaatacgtcgccaatttgacgacgaccgcgtcgaagcgatggaaaggagcagtggctaac

SEQ ID NO: 12 - protein, 343 aa, Nectria sp
MRLLPALSVVGVASAASIKSYLHAFEERAVTVTSQNLANFKFYVQHATAAYCNYDRAAGALISCSSNCPSIESNAAKIVGSFGGEDTGIAGYVSTDATRKEIVVSIRGSINVRNWITNLDFVWSSCSDLSSNCKAHAGFKDAWDEISTAAKAAVVSAKKANPSYTIVATGHSLGGAVATLAAAYIRAAGYSVDLYTFGSPRVGNDYFANFVTSQAGAEYRVTHLDDPVPRLPPILFGYRHTSPEYWLSNGGATTTTYSLSDIVVCEGIANTDCNAGTLGLDIIAHLIYFQDTSACNTGFTWKRDTLSDAELEEMVNKWAEQDVEYVANLTTTASKRWKGAVAN

SEQ ID NO: 13 - DNA, 999 bp, Fusarium sp
  CDS (1)..(999)
atgatgctcgtcctatcttttctctccataattgcctttgcggcagctagcccagtgccctccattgatgagaatactcaggtacttgagcatcgagctgtgacagtcacgacacaggacctgtcaaacttcaggttttatctccagcatgctgatgctgcgtattgcaatttcaatacggcagttggcaaaccagtccactgtggtgccgggaactgccctgatattgaaaaggacgctgccatcgttgtcggatcggtagttggtacgaagacgggcatcggtgcgtatgtggcaactgacaacgctcgtaaggagatcgttgtgtctgtgcgtggcagcatcaacgtgcgaaactggatcacaaacttcaactttggtcaaaagacctgcgatctcgttgctggctgcggggttcacaccggcttcttggacgcttgggaggaggttgcagccaatatcaaagctgctgtctcctcagcgaagactgcaaacccgactttcaagttcgtcgttaccggacactccctcggtggtgccgtcgctactgtcgcggctgcgtacctgcgcaaagacggctttccttttgacctctacacctacggctccccaagagttggaaacgactttttcgccaacttcgtcacccaacagacgggcgctgaatatcgcgtcacgcatggtgatgaccccgtcccacgtcttcctcccatcgtctttggataccgtcatactagcccagagtactggcttgacggtggcccactcgataaggactacaccgtgagcgagatcaaggtttgtgagggcattgcgaacgtaatgtgcaatggtggcacaataggtctggacattcttgcgcacatcacctatttccagagcatggccacttgtgcgccaatcgccatcccatggaagagggacatgtcagatgaggagttggacaagaagttgactcaatatagcgagatggatcaagaatttgttaagcagatgact

SEQ ID NO: 14 - protein, 333 aa, Fusarium sp
MMLVLSFLSIIAFAAASPVPSIDENTQVLEHRAVTVTTQDLSNFRFYLQHADAAYCNFNTAVGKPVHCGAGNCPDIEKDAAIVVGSVVGTKTGIGAYVATDNARKEIVVSVRGSINVRNWITNFNFGQKTCDLVAGCGVHTGFLDAWEEVAANIKAAVSSAKTANPTFKFVVTGHSLGGAVATVAAAYLRKDGFPFDLYTYGSPRVGNDFFANFVTQQTGAEYRVTHGDDPVPRLPPIVFGYRHTSPEYWLDGGPLDKDYTVSEIKVCEGIANVMCNGGTIGLDILAHITYFQSMATCAPIAIPWKRDMSDEELDKKLTQYSEMDQEFVKQMT

SEQ ID NO: 15 - DNA, 1047 bp, Gibberella zeae
  CDS (1)..(1047)
atgcgtctcctgtcactcctctcagttgtcacccttgcagtagccagccctctgagcgttgaagaatacgccaaggctctcgatgaacgagctgtctctgtctccaccaccgactttggcaacttcaagttctacatccagcacggcgccgcagcatactgcaactccgaagccccggccggtgcaaaggtcacctgcagcggaaacggctgtccaactgttcagtccaacggtgctaccatcgtggcatccttcactggatccaagactggaattggcggctacgtcgctacagaccctacacgcaaggagatcgtcgtctcgttccgtggtagcatcaacatccgcaactggcttaccaacctcgacttcgaccaggacgactgcagcctgacctcgggctgtggtgttcactcaggcttccagaatgcctggaacgagatctcagccgcagcaaccgccgctgtcgcaaaggcccgcaaggcaaacccttcgttcaaggtcgtctccgtaggtcactccctgggtggtgctgtagctacactggcaggcgcgaatctgcgaattggtggaacaccccttgacatctacacctacggttcaccccgagttggaaacacacagctcgctgcctttgtctcgaaccaggctggtggagagttccgcgttacgaacgccaaggaccccgtgcctcgtctcccccctctgatctttggataccgacacacatcccccgagtactggctgtctggcagcggaggtgacaagatcgactacaccatcaacgatgtcaaggtctgtgagggtgccgccaacctccagtgcaacggtggaacactcggattggatatcgatgcccatctccactacttccaggcaaccgatgcttgctctgctggcggcatctcgtggagaagatacaggagtgccaagcgtgagagcatctcagagagggctaccatgaccgatgccgagctcgagaagaagcttaacagctatgttgagatggataaggagtatatcaagactcacgccagccgctcatca

SEQ ID NO: 16 - protein, 349 aa, Gibberella zeae
MRLLSLLSVVTLAVASPLSVEEYAKALDERAVSVSTTDFGNFKFYIQHGAAAYCNSEAPAGAKVTCSGNGCPTVQSNGATIVASFTGSKTGIGGYVATDPTRKEIVVSFRGSINIRNWLTNLDFDQDDCSLTSGCGVHSGFQNAWNEISAAATAAVAKARKANPSFKVVSVGHSLGGAVATLAGANLRIGGTPLDIYTYGSPRVGNTQLAAFVSNQAGGEFRVTNAKDPVPRLPPLIFGYRHTSPEYWLSGSGGDKIDYTINDVKVCEGAANLQCNGGTLGLDIDAHLHYFQATDACSAGGISWRRYRSAKRESISERATMTDAELEKKLNSYVEMDKEYIKTHASRSS

SEQ ID NO: 17 - DNA, 45 bp, artificial sequence
  Primer 220506L1rev
gcagtatgctgcggcgccgtgctggacgtagaacttgaagtttgc

SEQ ID NO: 18 - DNA, 46 bp, artificial sequence
  Primer 220506L1fwp
cagcacggcgccgcagcatactgcaactacgaccgcgcagctggag

SEQ ID NO: 19 - DNA, 47 bp, artificial sequence
  Primer 220506L2rev
actgccacggatagagacgacaatctccttgcgagttgcgtcagttg

SEQ ID NO: 20 - DNA, 45 bp, artificial sequence
  Primer 220506L2fwp
attgtcgtctctatccgtggcagtattaacgtccgcaactggatc

SEQ ID NO: 21 - DNA, 45 bp, artificial sequence
  Primer 220506L3rev
tgcaccacccaagctatgtccggtggcgacgatggtgtagcttgg

SEQ ID NO: 22 - DNA, 48 bp, artificial sequence
  Primer 220506L3fwp
accggacatagcttgggtggtgcagttgctaccttagcagctgcttac

SEQ ID NO: 23 - DNA, 41 bp, artificial sequence
  Primer 220506L4rev
gtttccaactcggggtgaaccgaacgtgtacagatcgacac

SEQ ID NO: 24 - DNA, 42 bp, artificial sequence
  Primer 220506L4fwp
ggttcaccccgagttggaaacgactacttcgccaacttcgtc

SEQ ID NO: 25 - DNA, 42 bp, artificial sequence
  Primer 220506L5rev
gggtggaagacgaggaacagggtcgtcgaggtgtgtcacgcg

SEQ ID NO: 26 - DNA, 44 bp, artificial sequence
  Primer 220506L5fwp
cctgttcctcgtcttccacccatcctctttggctaccgtcatac

SEQ ID NO: 27 - DNA, 45 bp, artificial sequence
  Primer 220506L6rev
gaggtgggcaataatatcaaggccaagcgtgccggcattgcagtc

SEQ ID NO: 28 - DNA, 45 bp, artificial sequence
  Primer 220506L6fwp
ggccttgatattattgcccacctcatatacttccaggatacttcg

SEQ ID NO: 29 - DNA, 29 bp, artificial sequence
  Primer 230506L1
ggcaagcttccgccaggtgtcagtcaccc

SEQ ID NO: 30 - DNA, 46 bp, artificial sequence
  Primer 220506L7rev
gcagtatgctgcggcgccgtgctggagataaaacctgaagtttgac

SEQ ID NO: 31 - DNA, 45 bp, artificial sequence
  Primer 220506L7fwp
cagcacggcgccgcagcatactgcaatttcaatacggcagttggc

SEQ ID NO: 32 - DNA, 47 bp, artificial sequence
  Primer 220506L8rev
actgccacggatagagacgacaatctccttacgagcgttgtcagttg

SEQ ID NO: 33 - DNA, 45 bp, artificial sequence
  Primer 220506L8fwp
attgtcgtctctatccgtggcagtatcaacgtgcgaaactggatc

SEQ ID NO: 34 - DNA, 45 bp, artificial sequence
  Primer 220506L9rev
tgcaccacccaagctatgtccggtaacgacgaacttgaaagtcgg

SEQ ID NO: 35 - DNA, 45 bp, artificial sequence
  Primer 220506L9fwp
accggacatagcttgggtggtgcagtcgctactgtcgcggctgcg

SEQ ID NO: 36 - DNA, 42 bp, artificial sequence
  Primer 220506L10rev
gtttccaactcggggtgaaccgtaggtgtagaggtcaaaagg

SEQ ID NO: 37 - DNA, 42 bp, artificial sequence
  Primer 220506L10fwp
ggttcaccccgagttggaaacgactttttcgccaacttcgtc

SEQ ID NO: 38 - DNA, 42 bp, artificial sequence
  Primer 220506L11rev
gggtggaagacgaggaacagggtcatcaccatgcgtgacgcg

SEQ ID NO: 39 - DNA, 44 bp, artificial sequence
  Primer 220506L11fwp
cctgttcctcgtcttccacccatcgtctttggataccgtcatac

SEQ ID NO: 40 - DNA, 43 bp, artificial sequence
  Primer 220506L12rev
gaggtgggcaataatatcaaggcctattgtgccaccattgcac

SEQ ID NO: 41 - DNA, 45 bp, artificial sequence
  Primer 220506L12fwp
ggccttgatattattgcccacctcacctatttccagagcatggcc

SEQ ID NO: 42 - DNA, 43 bp, artificial sequence
  Primer 220506L13rev
gcagtatgctgcggcgccgtgctgtgcaaagagattgaactgg

SEQ ID NO: 43 - DNA, 46 bp, artificial sequence
  Primer 220506L13fwp
cagcacggcgccgcagcatactgcggaaaaaacaatgatgccccag

SEQ ID NO: 44 - DNA, 45 bp, artificial sequence
  Primer 220506L14rev
actgccacggatagagacgacaatcaatttgttcgtgttgtcgag

SEQ ID NO: 45 - DNA, 45 bp, artificial sequence
  Primer 220506L14fwp
attgtcgtctctatccgtggcagtcgttccatagagaactggatc

SEQ ID NO: 46 - DNA, 45 bp, artificial sequence
  Primer 220506L15rev
tgcaccacccaagctatgtccggtaaacaccacgcgatagtcggg

SEQ ID NO: 47 - DNA, 46 bp, artificial sequence
  Primer 220506L15fwp
accggacatagcttgggtggtgcattggcaactgttgccggagcag

SEQ ID NO: 48 - DNA, 42 bp, artificial sequence
  Primer 220506L16rev
gtttccaactcggggtgaaccatatgaaaacacgtcgatatc

SEQ ID NO: 49 - DNA, 42 bp, artificial sequence
  Primer 220506L16fwp
ggttcaccccgagttggaaacagggcttttgcagaattcctg

SEQ ID NO: 50 - DNA, 42 bp, artificial sequence
  Primer 220506L17rev
gggtggaagacgaggaacaggatcattggtgtgggtaatgcg

SEQ ID NO: 51 - DNA, 44 bp, artificial sequence
  Primer 220506L17fwp
cctgttcctcgtcttccaccccgcgaattcggttacagccattc

SEQ ID NO: 52 - DNA, 45 bp, artificial sequence
  Primer 220506L18rev
gaggtgggcaataatatcaaggccgttaggctggttattgccgcc

SEQ ID NO: 53 - DNA, 45 bp, artificial sequence
  Primer 220506L18fwp
ggccttgatattattgcccacctctggtacttcgggttaattggg

SEQ ID NO: 54 - DNA, 32 bp, artificial sequence
  Primer 230506L2
ggcggatccatatgcgtctcctgtcactcctc

SEQ ID NO: 55 - DNA, 45 bp, artificial sequence
  Primer 220506L19rev
gcagtatgctgcggcgccgtgctggatgtagaacttgaagttgcc

SEQ ID NO: 56 - DNA, 46 bp, artificial sequence
  Primer 220506L19fwp
cagcacggcgccgcagcatactgcaactccgaagccccggccggtg

SEQ ID NO: 57 - DNA, 47 bp, artificial sequence
  Primer 220506L20rev
actgccacggatagagacgacaatctccttgcgtgtagggtctgtag

SEQ ID NO: 58 - DNA, 47 bp, artificial sequence
  Primer 220506L20fwp
attgtcgtctctatccgtggcagtatcaacatccgcaactggcttac

SEQ ID NO: 59 - DNA, 45 bp, artificial sequence
  Primer 220506L21rev
tgcaccacccaagctatgtccggtggagacgaccttgaacgaagg

SEQ ID NO: 60 - DNA, 45 bp, artificial sequence
  Primer 220506L21fwp
accggacatagcttgggtggtgcagtagctacactggcaggcgcg

SEQ ID NO: 61 - DNA, 42 bp, artificial sequence
  Primer 220506L22rev
gtttccaactcggggtgaaccgtaggtgtagatgtcaagggg

SEQ ID NO: 62 - DNA, 42 bp, artificial sequence
  Primer 220506L22fwp
ggttcaccccgagttggaaacacacagctcgctgcctttgtc

SEQ ID NO: 63 - DNA, 42 bp, artificial sequence
  Primer 220506L23rev
gggtggaagacgaggaacagggtccttggcgttcgtaacgcg

SEQ ID NO: 64 - DNA, 42 bp, artificial sequence
  Primer 220506L23fwp
cctgttcctcgtcttccacccctgatctttggataccgacac

SEQ ID NO: 65 - DNA, 45 bp, artificial sequence
  Primer 220506L24rev
gaggtgggcaataatatcaaggccgagtgttccaccgttgcactg

SEQ ID NO: 66 - DNA, 46 bp, artificial sequence
  Primer 220506L24fwp
ggccttgatattattgcccacctccactacttccaggcaaccgatg

SEQ ID NO: 67 - DNA, 32 bp, artificial sequence
  Primer 230506L3
cttaagcttggctatgatgagcggctggcgtg